(19) United States
(12) Patent Application Publication
(10) Pub. No.: US 2012/0059424 A1
(43) Pub. Date: Mar. 8, 2012

(54) Clavicle Bone Plate

(75) Inventors: Scott Epperly, Torrance, CA (US); Raymond B. Raven, Glendale, CA (US)

(73) Assignee: ADVANCED ORTHOPAEDIC SOLUTIONS, INC., Torrance, CA (US)

(21) Appl. No.: 13/045,420

(22) Filed: Mar. 10, 2011

Related U.S. Application Data
(60) Provisional application No. 61/312,245, filed on Mar. 10, 2010.

(51) Int. Cl. A61B 17/80; A61B 17/88

(52) U.S. Cl. 606/281; 606/280; 606/286

(57) ABSTRACT

A bone plate for treating clavicle fractures. The bone plate may include an elongated body having one or more curved portions that roughly conform to the surface and/or anatomical curvature of the clavicle. One or more openings may be provided for receiving an anchoring member, such as a bone screw, for fastening at least a portion of the bone plate onto the clavicle. The bone plate may provide at least one rail member extending along a portion of the body of the bone plate. The bone plate, when affixed to the clavicle, permits the rail member to rest upon the outer surface of the clavicle, thus providing an area defining a cavity formed between an outer surface of the clavicle and a bottom surface of the body of the bone plate, and thus allowing blood to circulate against and around the clavicle.

### CROSS-REFERENCE TO RELATED APPLICATIONS

This application claims the benefit of U.S. Provisional Application Ser. No. 61/312,245 filed Mar. 10, 2010 which is now expired.

### BACKGROUND OF THE INVENTION

1. Field of the Invention

The present disclosure relates generally to bone treatment devices, and more particularly to bone plates for stabilizing clavicle fractures.

2. Description of Related Art

The clavicle is a doubly curved short bone that connects the arm (i.e., the upper limb) to the body (i.e., the trunk), located directly above the first rib and is often referred to as a collarbone. Unfortunately, clavicle fractures are common occurrences. Clavicle fractures, that is a broken collarbone, can occur in children and adolescents, athletes, or during many types of accidents and falls. Often clavicle fractures are treated using a plate-and-screw fixation or intramedullary devices. The plate-and-screw devices typically include a flat steel bone plate that is affixed along the superior aspect of the collar bone via several screws. In some cases, the plate may be removed after healing, but this is rarely required (e.g., based on nerve interaction or tissue aggravation), and typically considered an elective procedure.

A drawback of some of the known clavicle bone plates is that they are typically composed of relatively thick metallic plates that, after being attached to the fractured clavicle, may be clearly visible against the underside of the skin of the patient. This outcome is not aesthetically pleasing, especially for individuals that need to expose their upper chest areas (e.g., models, actors, swimmers, divers, and/or the like) for vocational or professional purposes. Additionally, the entire support surface of many of the known clavicle bone plates, after being attached, contact the clavicle, thus lessening the amount of blood, and correspondingly the oxygen and other bone nutrients contained therein, that flows over the clavicle, especially in the area of the fracture. This situation may potentially delay the healing of the fracture, as well as impair the overall health of the clavicle itself.

The features and advantages of the disclosure will be set forth in the description which follows, and in part will be apparent from the description, or may be learned by the practice of the disclosure without undue experimentation. The features and advantages of the disclosure may be realized and obtained by means of the instruments and combinations particularly pointed out in the appended claims.

### SUMMARY OF THE INVENTION

A bone plate for affixation to a bone for treating bone fractures, especially clavicle fractures. The clavicle bone plate may include an elongate body having a first end portion at a proximal end and a second end portion toward a distal end. The clavicle bone plate may include one or more curved portions that roughly conform to the surface and/or anatomical curvature of the clavicle, especially the surface and/or anatomical curvature of the shaft of the clavicle. One or more openings may be provided in the body, the first end portion, and/or the second end portion for receiving an anchoring member, such as a bone screw, for fastening at least a portion of the clavicle bone plate onto the clavicle, especially in proximity to the fracture site. The clavicle bone plate may also provide at least one, and more preferably at least two spaced and opposed rail members extending along at least a portion of the body of the clavicle bone plate. The rail member may provide enhanced durability and strength to the clavicle bone plate, while also permitting the reduction of the thickness of a central portion extending along the length of the clavicle bone plate, thereby reducing the overall thickness, weight, and cost of the material needed to form the clavicle bone plate. The clavicle bone plate, when affixed to the clavicle, permits the rail member to rest upon the outer surface of the clavicle. The clavicle bone plate thus may provide an area defining a cavity formed between an outer surface of the clavicle and a bottom surface of the body of the clavicle bone plate, thus allowing blood to circulate against and around the clavicle, especially in proximity to the fracture site.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The features and advantages of the disclosure will become apparent from a consideration of the subsequent detailed description presented in connection with the accompanying drawings in which:

FIG. 1 is a top plan view of a bone plate in accordance with the general teachings of the present invention;

FIG. 2 is a sectional view taken along line A-A of FIG. 1;

FIG. 3 is a sectional view taken along line B-B of FIG. 1;

FIG. 4 is a sectional view taken along line C-C of FIG. 1;

FIG. 5 is a side elevational view of the bone plate depicted in FIG. 1;

FIG. 6 is a perspective view of the bone plate depicted in FIG. 1 affixed to a superior aspect of a clavicle bone;

FIG. 7 is a sectional view taken along line D-D of FIG. 6;

FIG. 8 is a top plan view of a bone plate in accordance with a first alternative embodiment of the present invention;

FIG. 9 is a sectional view taken along line E-E of FIG. 8;

FIG. 10 is a sectional view taken along line F-F of FIG. 8;

FIG. 11 is a side elevational view of the bone plate depicted in FIG. 8;

FIG. 12 is a top plan view of a bone plate in accordance with a second alternative embodiment of the present invention;

FIG. 13 is a side elevational view of the bone plate depicted in FIG. 12;

FIG. 14 is a bottom perspective view of the bone plate depicted in FIG. 12;

FIG. 15 is a top plan view of a bone plate in accordance with a third alternative embodiment of the present invention;

FIG. 16 is a sectional view taken along line G-G of FIG. 15;

FIG. 17 is a sectional view taken along line H-H of FIG. 15;

FIG. 18 is a sectional view taken along line I-I of FIG. 15;

FIG. 19 is a sectional view taken along line J-J of FIG. 15;

FIG. 20 is a sectional view taken along line K-K of FIG. 15;

FIG. 21 is a perspective view of the bone plate depicted in FIG. 15;

FIG. 22 is a front elevational view of a bone plate affixed to an anterior aspect of a bone in accordance with a fourth alternative embodiment of the present invention;

FIG. 23 is a top plan view of the bone plate depicted in FIG. 22;

FIG. 24 is a bottom plan view of a bone plate in accordance with a fifth alternative embodiment of the present invention;

FIG. 25 is a perspective view of a bone plate affixed to a superior aspect of a clavicle bone in accordance with a sixth alternative of the present invention;

FIG. 26A is a bottom perspective view of a bone plate in accordance with a seventh alternative embodiment of the present invention;

FIG. 26B is a bottom perspective view of a bone plate in accordance with an eighth alternative embodiment of the present invention;

FIG. 26C is a bottom perspective view of a bone plate in accordance with a ninth alternative embodiment of the present invention; and

FIG. 27 is a perspective view of the bone plate depicted in FIG. 26B affixed to an anterior aspect of a clavicle bone in accordance with a tenth alternative embodiment of the present invention.

### DETAILED DESCRIPTION OF THE INVENTION

The following detailed description of the invention is merely exemplary in nature and is in no way intended to limit the invention, its application, or uses.

For the purposes of promoting an understanding of these principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art having possession of this disclosure, are to be considered within the scope of the disclosure claimed.

Before the present apparatus and methods for treating a bone fracture are disclosed and described, it is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present disclosure will be limited only by the appended claims and equivalents thereof.

It must be noted that, as used in this specification and the appended claims, the singular forms “a,” “an,” and “the” include plural referents unless the context clearly dictates otherwise. Moreover, as used herein, the terms “comprising”, “including”, “containing”, “characterized by” and grammatical equivalents thereof are inclusive or open-ended terms that do not exclude additional, unrecited elements or method steps. In addition, the term “at” when referring to the location or placement of an element or object means in, near or by the area or location occupied by the particular structure or element referred to.

It must be noted that, as used in this specification and the appended claims, the terms “top,” “bottom,” “side,” “end,” “left,” “right,” and/or the like, are used herein solely for reference purposes and are not meant to limit the scope of the present invention.

As used herein, the term “proximal” shall refer broadly to the concept of a nearest portion.

As used herein, the term “distal” shall generally refer to the opposite of proximal, and thus to the concept of a further portion, or a furthest portion, depending upon the context.

As used herein, the term “anterior” shall refer broadly to the concept of the front side of the body.

As used herein, the term “superior” shall refer broadly to the concept of the portion of the body closest to the head.

As used herein, the term “acromial” shall refer broadly to the concept of area of the clavicle nearest to or proximate the acromion.

As used herein, the term “sternal” shall refer broadly to the concept of area of the clavicle nearest to or proximate the sternum.

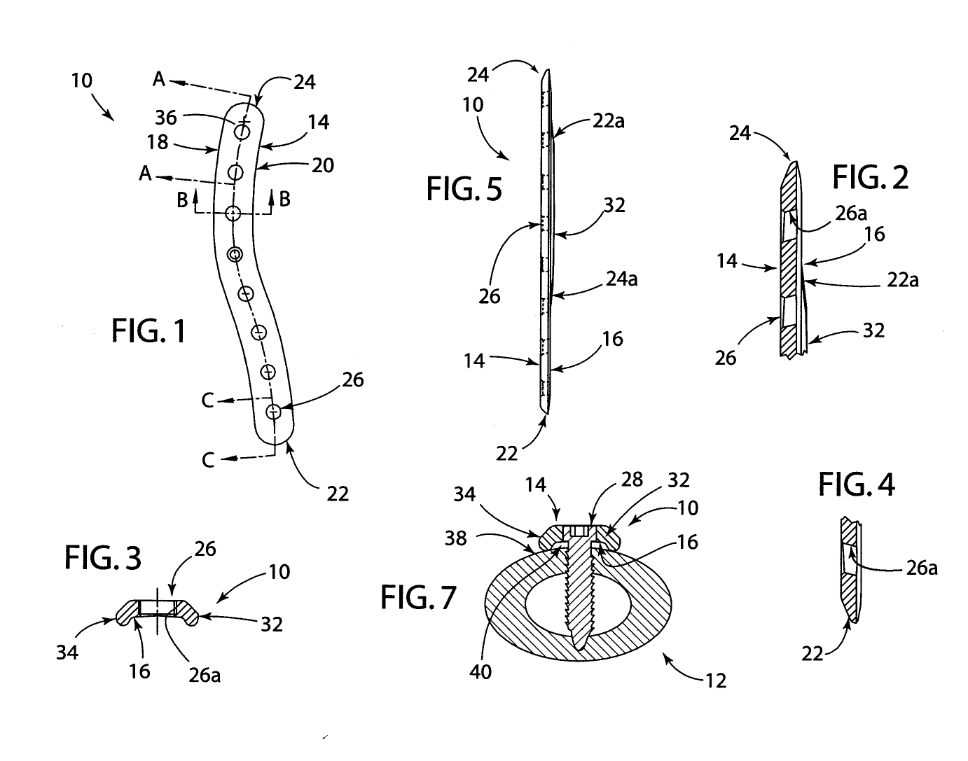

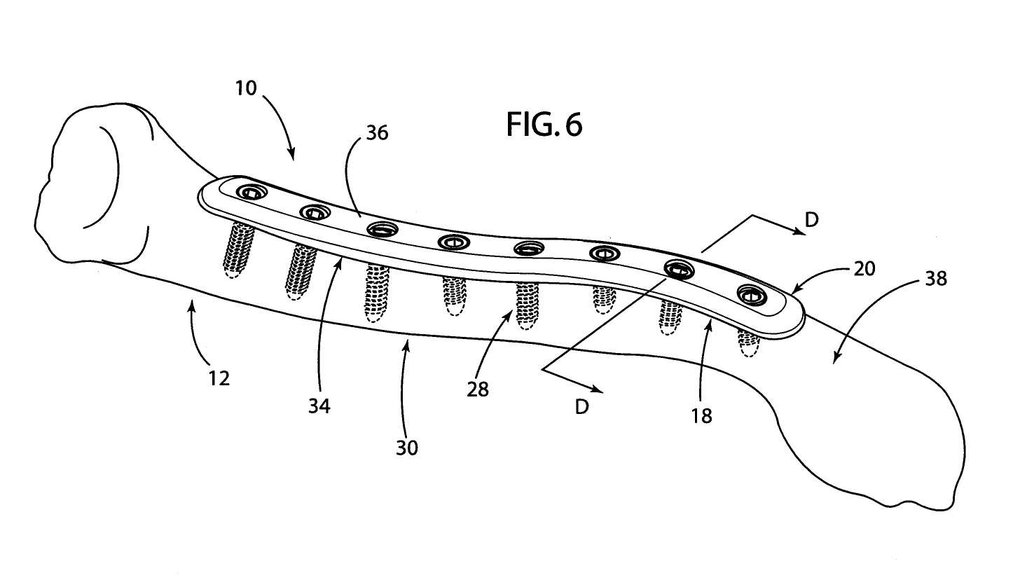

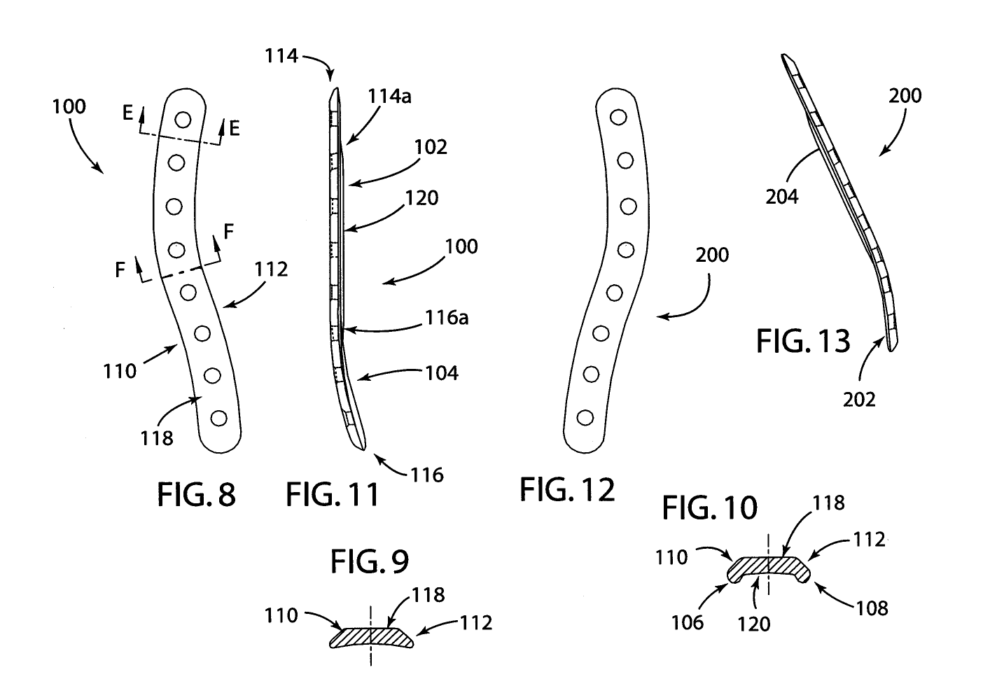

Referring now to FIGS. 1-7, a bone plate is generally shown at 10. The bone plate 10 is primarily intended for affixation to a fractured clavicle 12; however, it should be appreciated that the bone plate 10 may be used to repair fractures of other bones, including those of humans and/or other mammals.

The bone plate 10 includes a top surface 14, a bottom surface 16, a first side surface 18 and a spaced and an opposed second side surface 20, and a first end portion 22 and a spaced and opposed second end portion 24. At least one, and more preferably, a plurality of through bores 26 are formed which extend from the top surface 14 to the bottom surface 16. The walls 26a of the through bores 26 can be substantially vertical, see FIG. 3, and/or angled, see FIGS. 2 and 4. The walls 26a of the through bores 26, which are shown as being smooth, are intended to receive anchoring or fastening members 28, which may include bone screws and/or the like. It should be appreciated that the walls 26a of the through bores 26 can also be threaded.

FIGS. 1 and 6 illustrate the bone plate 10, in a top plan view, as being curved so as to mimic or correspond to the anatomical curvature of a shaft portion 30 of the clavicle 12, for example. FIG. 5 shows the bone plate 10, in a side elevational view, as being substantially planar so as to mimic or correspond to a substantially planar bone surface of the shaft portion 30 of the clavicle 12. In this case, the bone plate 10 is very suitable for affixation to a bone, such as a clavicle, that has an anatomical curvature in one plane, while also having a substantially planar portion in another plane. For example, the bone plate 10 would be very suitable for affixation to the area proximate to the midpoint of the shaft portion 30 of the clavicle 12, on either an anterior or superior aspect thereof.

Referring specifically to FIGS. 2-5, and in accordance with one aspect of this embodiment, at least one, and more preferably, at least two rail members 32, 34 are provided for on or near the first and second side surfaces 18, 20, respectively. The rail members 32, 34 extend from an inboard point 22a proximate to a first end portion 22, which may be substantially planar, to an inboard point 24a proximate to the second end portion 24, which may also be substantially planar. As shown, the rails members 32, 34 need not extend the entire length of the bone plate 10; however, it is contemplated that the rails members 32, 34, may extend the entire length of the bone plate 10. The rail members 32, 34, may be integral to the body 36 of the bone plate 10; however, the rail members 32, 34, may be attached through a suitable process such as fastening, welding, gluing, and/or the like at a later time after initial formation of the body 36 of the bone plate 10. In the present embodiment the rail members 32, 34, provide enhanced durability and strength to the bone plate 10. Additionally, use of the rail members 32, 34 enables configuration of the bone plate 10 in a manner that reduces the thickness of a central portion extending along the length of the bone plate 10, thereby reducing the overall thickness, weight, and cost of the material needed to form the bone plate 10.

Turning to FIGS. 6 and 7, the rail members 32, 34, respectively, enable the bone plate 10, when affixed to a bone, such as the clavicle 12, to maintain at least a portion of the bottom surface 16 above at least a portion of the outer surface 38 of the bone, so as to define a cavity 40 therebetween. Furthermore, it is understood that the cavity 40 forms a gap or passageway enabling blood flow or circulation against and around the clavicle 12, especially in proximity to the fracture site so as to enable and/or speed the healing process of the fracture(s).

With respect to affixation of the bone plate 10 to the bone, it is known in the art; i.e., once the bone plate 10 is properly positioned on the bone, to insert at least one of the fastening members 28 through at least one of the through bores 26 of the bone plate 10, whereupon the fastening member 28 is screwed, or otherwise driven, into the bone, in this case, the clavicle 12.

Referring now to FIGS. 8-11, a first alternative bone plate is generally shown at 100. As with the embodiment depicted in FIGS. 1-7, the bone plate 100 is primarily intended for affixation to a fractured clavicle 12; however, it should be appreciated that the bone plate 100 may be used to repair fractures of other bones.

FIG. 8 shows the bone plate 100, in a top plan view, as being curved so as to mimic or correspond to the anatomical curvature of a shaft portion 30 of the clavicle 12. FIG. 11 shows the bone plate 100, in a side elevational view, as including a first portion 102 being substantially planar and a second portion 104 being curved so as to mimic or correspond to a substantially curved bone surface of the shaft portion 30 of the clavicle 12. In this case, the bone plate 100 is very suitable for affixation to a bone, such as a clavicle, that has an anatomical curvature in one plane, while also having an anatomical curvature in another plane. Accordingly, the bone plate 100 is suitable for affixation to the area proximate to the acromial and/or sternal portions of the clavicle 12, e.g., on either an anterior or superior aspect thereof.

Referring specifically to FIGS. 9 and 10, and in accordance with one aspect of this embodiment, at least one, and more preferably, two rail members 106, 108 are provided for on or near side surfaces 110, 112. The rail members 106, 108 extend from an inboard point 114a proximate a first end portion 114, which may be substantially planar, to an inboard point 116a proximate to the relatively thick or wide second end portion 116, which may also be substantially planar. That is, the rails members 106, 108 do not necessarily extend the entire length of the bone plate 100; however, it should be appreciated that the rails members 106, 108 may extend the entire length of the bone plate 100. The rail members 106, 108 may be integral to the body 118 of the bone plate 100; however, it should be appreciated that the rail members 106, 108 may be attached by fastening, welding, gluing, and/or the like at a later time after initial formation of the body 118 of the bone plate 100. The rail members 106, 108 provide enhanced durability and strength to the bone plate 100. Additionally, use of the rail members 106, 108 enables configuration of the bone plate 100 in a manner that reduces the thickness of a central portion extending along the length of the bone plate 100, thereby reducing the overall thickness, weight, and cost of the material needed to form the bone plate 100.

Referring specifically to FIG. 10, the rail members 106, 108 enable the bone plate 100, when affixed to a bone, such as the clavicle 12, to maintain at least a portion of the bottom surface 120 above at least a portion of the outer surface of the bone, such as the clavicle 12, so as to define a cavity 40 therebetween, see FIG. 7. Furthermore, the cavity 40 forms a gap or passageway enabling blood flow or circulation against and around the clavicle 12, especially in proximity to the fracture site so as enable and/or speed the healing process of the fracture(s).

Referring now to FIGS. 12-14, a second alternative bone plate is generally shown at 200. As with the embodiments depicted in FIGS. 1-11, the bone plate 200 is primarily intended for affixation to a fractured clavicle 12; however, it should be appreciated that the bone plate 200 may be used to repair fractures of other bones.

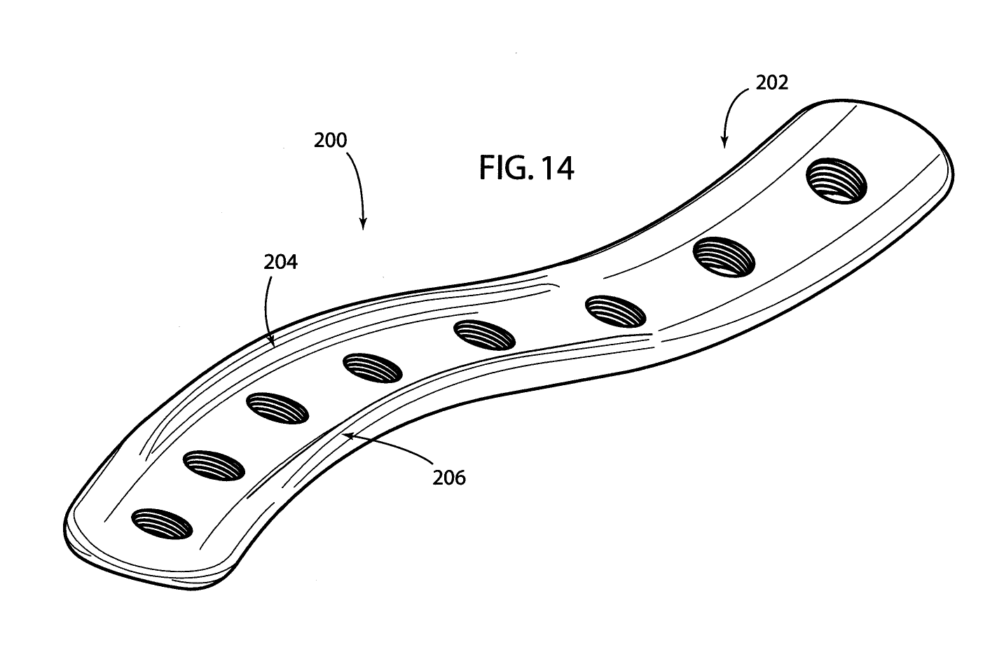

The primary difference between the embodiments depicted in FIGS. 8-11 and the embodiment depicted in FIGS. 12-14 is that the second end portion 202 shown in FIG. 13 has a relatively thinner or narrower profile than that of the second end portion 116 shown in FIG. 11. Referring specifically to FIG. 14, the rail members 204, 206 are clearly seen.

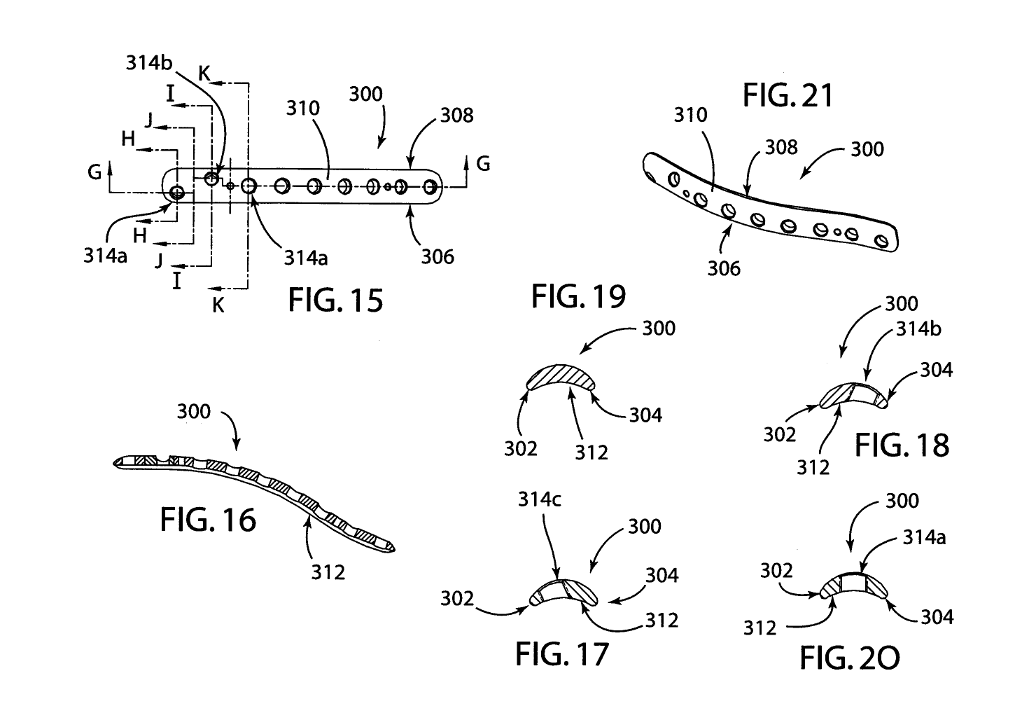

Referring now to FIGS. 15-21, a third alternative bone plate is generally shown at 300. As with the embodiments depicted in FIGS. 1-14, the bone plate 300 is primarily intended for affixation to a fractured clavicle 12; however, it should be appreciated that the bone plate 300 may be used to repair fractures of other bones.

The primary difference between the embodiments depicted in FIGS. 1-14 and the embodiment depicted in FIGS. 15-21 is that the bone plate 300 is substantially straight in the top plan view but also has a curvature in the sectional views, see FIGS. 16, 19 and 21, so as to mimic or correspond to both an anatomical curvature and a curved bone surface of the shaft portion 30 of the clavicle 12. By way of example, the bone plate 300 would be very suitable for affixation to the area proximate to the midpoint of the shaft portion 30 of the clavicle 12, e.g., on an anterior aspect thereof.

Referring specifically to FIGS. 17-20, and in accordance with one aspect of this embodiment, at least one, and more preferably, two rail members 302, 304 are provided for on or near side surfaces 306, 308. The rail members 302, 304 may extend the entire length of the bone plate 300. The rail members 302, 304 may be integral to the body 310 of the bone plate 300; however, it is understood that the rail members 302, 304 may be attached by fastening, welding, gluing, and/or the like at a later time after initial formation of the body 310 of the bone plate 300. The rail members 302, 304 may provide enhanced durability and strength to the bone plate 300. Additionally, the rail members 302, 304 may provide a bone plate 300 configuration that reduces the thickness of a central portion extending along the length of the bone plate 300, thereby reducing the overall thickness, weight, and cost of the material needed to form the bone plate 300. As with the previously described embodiments, the rail members 302, 304 enable the bone plate 300, when affixed to a bone, such as the clavicle 12, to maintain at least a portion of the bottom surface 312 above at least a portion of the outer surface of the bone, such as the clavicle 12, so as to define a cavity 40 therebetween, see FIG. 7. Furthermore, the cavity 40 forms a gap or passageway enabling blood flow or circulation against and around the clavicle 12, especially in proximity to the fracture site so as enable and/or speed the healing process of the fracture(s).

Referring specifically to FIGS. 15, 17, 18 and 21, the through bores 314a, 314b, 314c provided in the bone plate 300 may be offset with respect to one another, i.e., arranged in a non-linear configuration, so as to allow the orthopedic surgeon the freedom to place fastening members 28, such as bone screws, in the precise location(s) indicated.

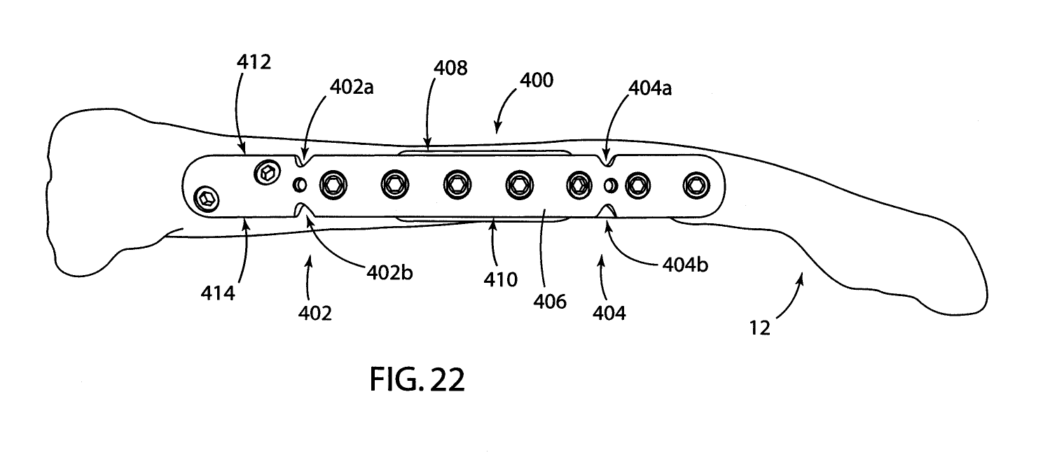

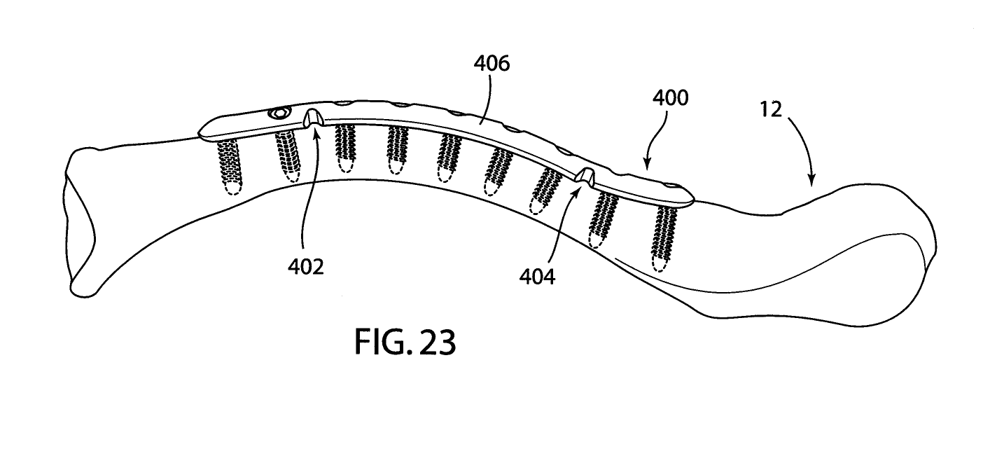

Referring now to FIGS. 22-23, a fourth alternative bone plate is generally shown at 400. As with the embodiments depicted in FIGS. 1-21, the bone plate 400 is primarily intended for affixation to a fractured clavicle 12; however, it should be appreciated that the bone plate 400 may be used to repair fractures of other bones.

The primary difference between the embodiments depicted in FIGS. 1-21 and the embodiment depicted in FIGS. 22-23 is that the bone plate 400 includes at least one, and preferably at least first and second pairs of notch portions 402, 404 including notches 402a and 402b and notches 404a and 404b, formed in the body 406 of the bone plate 400. These notch portion pairs 402, 404, respectively, allow the orthopedic surgeon the freedom to manipulate the bone plate 400, via an appropriate tool (not shown) so as to achieve an acceptable position of the bone plate 400 on the bone surface.

As with the previously described embodiments, and in accordance with one aspect of this embodiment, at least one, and more preferably, two rail members 408, 410 are provided for on or near side surfaces 412, 414. The rail members 408, 410 may extend the entire length of the bone plate 400. The rail members 408, 410 may be integral to the body 406 of the bone plate 400; however, it should be appreciated that the rail members 408, 410 may be attached by fastening, welding, gluing, and/or the like at a later time after initial formation of the body 406 of the bone plate 400. The rail members 408, 410 provide enhanced durability and strength to the bone plate 400. Additionally, the rail members 408, 410 provide a bone plate 400 that may be configured to reduce the thickness of a central portion extending along the length of the bone plate 400, thereby reducing the overall thickness, weight, and cost of the material needed to form the bone plate 400. As with the previously described embodiments, the rail members 408, 410 enable the bone plate 400, when affixed to a bone, such as the clavicle 12, to maintain at least a portion of the bottom surface above at least a portion of the outer surface of the bone, such as the clavicle 12, so as to define a cavity 40 therebetween, see FIG. 7. Furthermore, the cavity 40 forms a gap or passageway enabling blood flow or circulation against and around the clavicle 12, especially in proximity to the fracture site so as to enable and/or speed the healing process of the fracture(s).

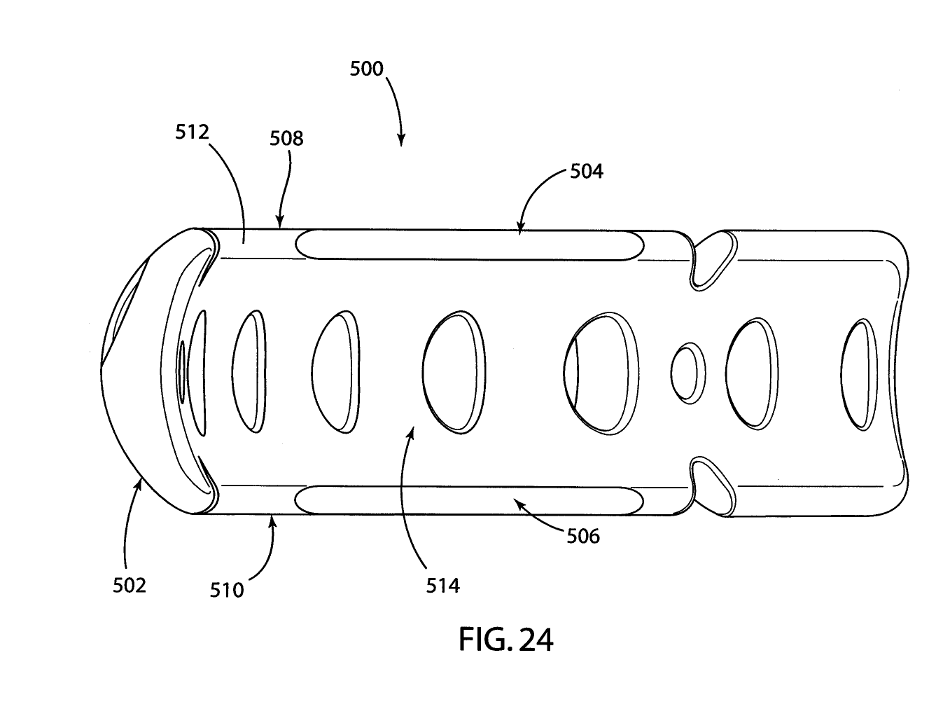

Referring now to FIG. 24, a fifth alternative bone plate is generally shown at 500. As with the embodiments depicted in FIGS. 1-23, the bone plate 500 is primarily intended for affixation to a fractured clavicle 12; however, it should be appreciated that the bone plate 500 may be used to repair fractures of other bones.

The primary difference between the embodiments depicted in FIGS. 1-23 and the embodiment depicted in FIG. 24 is that the bone plate 500 includes at least one portion 502 that is operable to extend to and curve over the acromial portion of the clavicle 12.

As with the previously described embodiments, and in accordance with one aspect of this embodiment, at least one, and more preferably, two rail members 504, 506 are provided for on or near side surfaces 508, 510. The rail members 504, 506 may extend the entire length of the bone plate 500. The rail members 504, 506 may be integral to the body 512 of the bone plate 500; however, it should be appreciated that the rail members 504, 506 may be attached by fastening, welding, gluing, and/or the like at a later time after initial formation of the body 512 of the bone plate 500. The rail members 504, 506 provide enhanced durability and strength to the bone plate 500. Additionally, the rail members 504, 506 provide a bone plate 500 that may be configured to reduce the thickness of a central portion extending along the length of the bone plate 500, thereby reducing the overall thickness, weight, and cost of the material needed to form the bone plate 500. As with the previously described embodiments, the rail members 504, 506, respectively, enable the bone plate 500, when affixed to a bone, such as the clavicle 12, to maintain at least a portion of the bottom surface 514 above at least a portion of the outer surface of the bone, such as the clavicle 12, so as to define a cavity 40 therebetween, see FIG. 7. Furthermore, the cavity 40 forms a gap or passageway enabling blood flow or circulation against and around the clavicle 12, especially in proximity to the fracture site so as to enable and/or speed the healing process of the fracture(s).

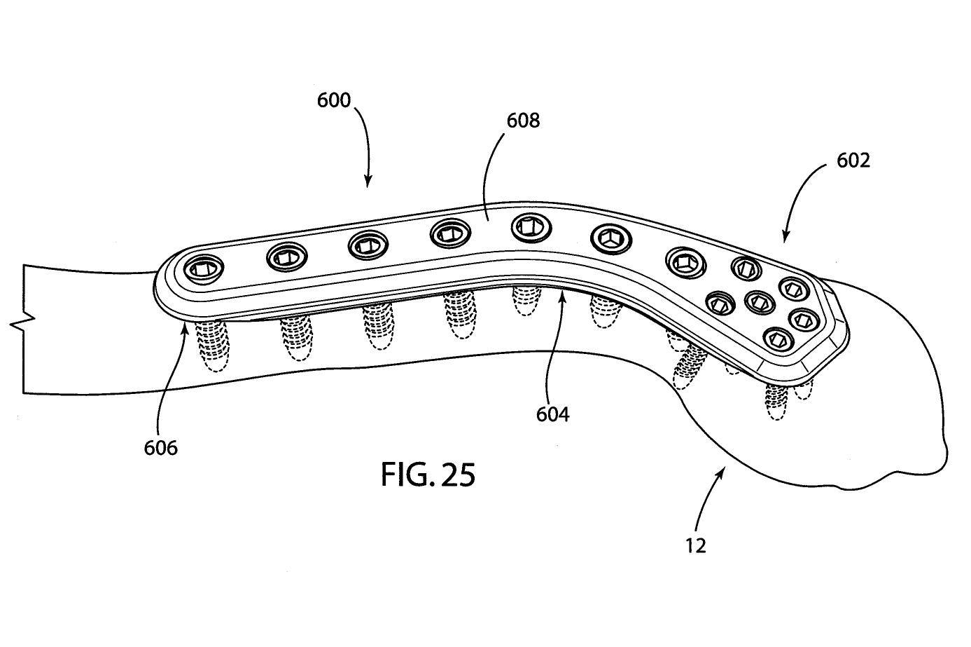

Referring now to FIG. 25, a sixth alternative bone plate is generally shown at 600. As with the embodiments depicted in FIGS. 1-24, the bone plate 600 is primarily intended for affixation to a fractured clavicle 12; however, it should be appreciated that the bone plate 600 may be used to repair fractures of other bones.

The primary difference between the embodiments depicted in FIGS. 1-24 and the embodiment depicted in FIG. 25 is that the bone plate 600 includes a widened acromial portion 602 that is operable to extend to and cover a substantial portion of the acromial portion of the clavicle 12.

As with the previously described embodiments, and in accordance with one aspect of this embodiment, at least one rail member 604 is provided for on or near at least one side surface 606. The rail member 604 may extend the entire length of the bone plate 600. The rail member 604 may be integral to the body 608 of the bone plate 600; however, it should be appreciated that the rail member 604 may be attached by fastening, welding, gluing, and/or the like at a later time after initial formation of the body 608 of the bone plate 600. The rail member 604 provides enhanced durability and strength to the bone plate 600. Additionally, the rail member 604 provides a bone plate 600 that may be configured to reduce the thickness of a central portion extending along the length of the bone plate 600, thereby reducing the overall thickness, weight, and cost of the material needed to form the bone plate 600. As with the previously described embodiments, the rail member 604 enables the bone plate 600, when affixed to a bone, such as the clavicle 12, to maintain at least a portion of the bottom surface above at least a portion of the outer surface of the bone, such as the clavicle 12, so as to define a cavity 40 therebetween, see FIG. 7. Furthermore, the cavity 40 forms a gap or passageway enabling blood flow or circulation against and around the clavicle 12, especially in proximity to the fracture site so as to enable and/or speed the healing process of the fracture(s).

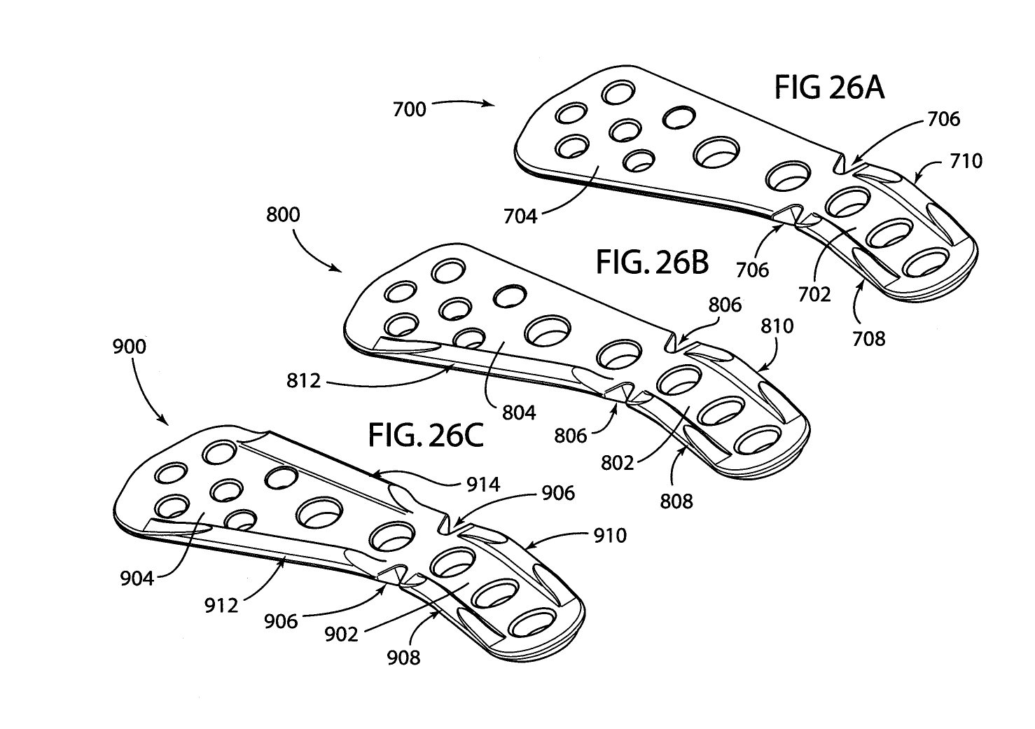

Referring now to FIGS. 26A, 26B and 26C, seventh, eighth, and ninth alternative bone plates are generally shown at 700, 800 and 900, respectively. As with the embodiments depicted in FIGS. 1-25, the bone plates 700, 800 and 900, respectively, are primarily intended for affixation to a fractured clavicle 12; however, it should be appreciated that the bone plates 700, 800 and 900 may be used to repair fractures of other bones.

The primary difference between the embodiments depicted in FIGS. 1-25 and the embodiment depicted in FIGS. 26A, 26B and 26C, is that the bone plates 700, 800 and 900 can include various rail member configurations. Referring specifically to FIG. 26A, bone plate 700 includes two major body portions 702, 704, respectively, separated by a notch pair 706, wherein portion 702 includes two spaced and opposed rail members 708, 710. Referring specifically to FIG. 26B, bone plate 800 includes two major body portions 802, 804 separated by a notch pair 806, wherein portion 802 includes two spaced and opposed rail members 808, 810 and portion 804 includes a single rail member 812. Referring specifically to FIG. 26C, bone plate 900 includes two major body portions 902, 904 separated by a notch pair 906, wherein portion 902 includes two spaced and opposed rail members 908, 910 and portion 904 also includes two spaced and opposed rail members 912, 914. In this manner, the orthopedic surgeon can choose from a number of configurations of bone plates to determine whether the presence of a rail member on a particular body portion of the bone plate is indicated.

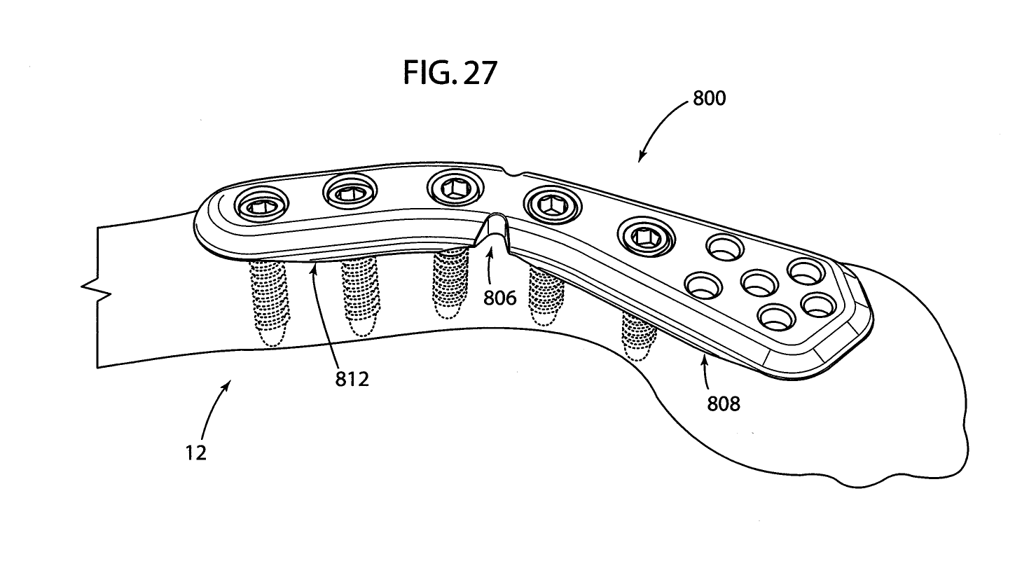

As with the previously described embodiments, and in accordance with one aspect of these embodiments, the respective rail members enable the bone plates 700, 800, 900 when affixed to a bone, see FIG. 27, such as the clavicle 12, to maintain at least a portion of the bottom surface above at least a portion of the outer surface of the bone, such as the clavicle 12, so as to define a cavity 40 there between, see FIG. 7. Furthermore, the cavity 40 forms a gap or passageway enabling blood flow or circulation against and around the clavicle 12, especially in proximity to the fracture site so as to enable and/or speed the healing process of the fracture(s).

It will be appreciated that the bone plates of the present invention may be formed of any known material capable of providing suitable strength and durability characteristics, as well as allowing for compatibility with the bone in which it is to be installed.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. A method of treating a fracture in a bone, comprising:
providing a bone plate having at least one rail member extending along a side surface of said bone plate; and
placing said rail member of said bone plate onto an outer surface of the bone;
wherein when said rail member of said bone plate is placed onto the outer surface of the bone, an area defining a cavity is formed between a bottom surface of said bone plate and the outer surface of the bone so as to allow a flow of fluid through said cavity.

2. The method of treating a fracture in a bone as set forth in claim 1, wherein said bone plate further comprises at least one opening formed therein.

3. The method of treating a fracture in a bone as set forth in claim 2, further comprising inserting at least one fastening member into said at least one opening in said bone plate.

4. The method of treating a fracture in a bone as set forth in claim 1, further comprising providing at least one curved portion to said bone plate.

5. The method of treating a fracture in a bone as set forth in claim 4, wherein said curved portion extends in a first dimensional plane.

6. The method of treating a fracture in a bone as set forth in claim 5, wherein said curved portion extends in a second dimensional plane.

7. The method of treating a fracture in a bone as set forth in claim 1, further comprising providing an area defining a notch formed in said bone plate.

8. The method of treating a fracture in a bone as set forth in claim 7, wherein said notch defines a first portion and a second portion of said bone plate.

9. The method of treating a fracture in a bone as set forth in claim 8, wherein a first portion of said rail member is positioned on said first portion of said bone plate and a second portion of said rail member is positioned on said second portion of said bone plate.

10. The method of treating a fracture in a bone as set forth in claim 1, wherein the bone is a clavicle.

11. A bone plate system for treating a fracture in a bone, comprising:
a bone plate member having at least one rail member extending along a side surface of said bone plate;
wherein said rail member of said bone plate is operable to be placed onto an outer surface of the bone;
wherein when said rail member of said bone plate is placed onto the outer surface of the bone, an area defining a cavity is formed between a bottom surface of said bone plate and the outer surface of the bone so as to allow a flow of fluid through said cavity.

12. A bone plate system for treating a fracture in a bone as set forth in claim 11, wherein said bone plate further comprises at least one opening formed therein.

13. A bone plate system for treating a fracture in a bone as set forth in claim 12, further comprising at least one fastening member operable to be inserted into said at least one opening in said bone plate.

14. A bone plate system for treating a fracture in a bone as set forth in claim 11, wherein said bone plate includes at least one curved portion formed therein.

15. A bone plate system for treating a fracture in a bone as set forth in claim 14, wherein said curved portion extends in a first dimensional plane.

16. A bone plate system for treating a fracture in a bone as set forth in claim 15, wherein said curved portion extends in a second dimensional plane.

17. A bone plate system for treating a fracture in a bone as set forth in claim 11, wherein an area defining a notch is formed in said bone plate.

18. A bone plate system for treating a fracture in a bone as set forth in claim 17, wherein said notch defines a first portion and a second portion of said bone plate.

19. A bone plate system for treating a fracture in a bone as set forth in claim 18, wherein a first portion of said rail member is positioned on said first portion of said bone plate and a second portion of said rail member is positioned on said second portion of said bone plate.

20. A bone plate system for treating a fracture in a bone as set forth in claim 11, wherein the bone is a clavicle.
